SEQUENCE LISTING (1 sequence)

SEQ ID NO: 1 - protein, 532 aa, Streptomyces rugosporus
MERRKRERLGSLGRPTKKELRMIRSVVIVGGGTAGWMTASYLKAAFDDRIDVTLVESGNVRRIGVGEATFSTVRHFFDYLGLDEREWLPRCAGGYKLGIRFENWSEPGEYFYHPFERLRVVDGFNMAEWWLAVGDRRTSFSEACYLTHRLCEAKRAPRMLDGSLFASQVDESLGRSTLAEQRAQFPYAYHFDADEVARYLSEYAIARGVRHVVDDVQHVGQDERGWISGVHTKQHGEISGDLFVDCTGFRGLLINQTLGGRFQSFSDVLPNNRAVALRVPRENDEDMRPYTTATAMSAGWMWTIPLFKRDGNGYVYSDEFISPEEAERELRSTVAPGRDDLEANHIQMRIGRNERTWINNCVAVGLSAAFVEPLESTGIFFIQHAIEQLVKHFPGERWDPVLISAYNERMAHMVDGVKEFLVLHYKGAQREDTPYWKAAKTRAMPDGLARKLELSASHLLDEQTIYPYYHGFETYSWITMNLGLGIVPERPRPALLHMDPAPALAEFERLRREGDELIAALPSCYEYLASIQ

(19) United States
(12) Patent Application Publication
(10) Pub. No.: US 2022/0340915 A1
(43) Pub. Date: Oct. 27, 2022

(54) METHOD TO ENABLE SOIL BACTERIA TO PRODUCE POWERFUL CHLORINATED AUXINS

(71) Applicant: Gary Zhan, North Logan, UT (US)

(72) Inventor: Gary Zhan, North Logan, UT (US)

(21) Appl. No.: 17/235,920

(22) Filed: Apr. 20, 2021

(51) Int. Cl. C12N 15/70; C12N 9/02

(52) U.S. Cl. CPC C12N 15/70; C12Y 114/19; C12N 9/0071

(57) ABSTRACT

The present invention comprises a novel method to engineer soil bacteria to produce powerful chlorinated auxins. While chlorinated auxins were only found in few plant species, this technology will allow the construction of soil bacterial strains capable of producing chlorinated derivatives of indole-3-acetic acid (IAA). A select halogenase can be expressed in soil bacteria by inserting it into the genome or through an expression vector. The engineered strains can then be applied to any plants to promote growth, thus having promising applications in agriculture.

### CROSS-REFERENCE TO RELATED APPLICATIONS

A provisional patent application (U.S. 63/013,367) has been previously filed for this invention on Apr. 21, 2020.

### REFERENCE TO A “SEQUENCE LISTING”

The amino acid sequence of a representative tryptophan halogenase, PyrH, is shown in sequence listing.

### BACKGROUND

According to the World Health Organization, the number of hungry people in the world reached 821 million in 2017, meaning that one in every nine people globally is suffering from food insecurity. Population increase and climate change will make the situation even more challenging. On top of that issue, the current agricultural practices, including the application of chemical fertilizers and pesticides, are environmentally unsustainable. Therefore, there is an urgent need to develop novel agricultural technologies to enhance global food production and lower the food costs in a sustainable way.

Soil bacteria are known to benefit crop growth in a variety of ways, such as producing indole-3-acetic acid (IAA), a native auxin that can stimulate plant growth and enhance crop yield/quality. The chlorinated derivatives of IAA, such as 4-chloro-IAA (4-CI-IAA) and 5-chloro-IAA (5-CI-IAA), have been reported to be much more active and thus are better plant growth promoters than IAA. However, chlorinated auxins are only present in very few plant species and no soil bacteria can produce these powerful plant growth stimulator, which has limited their efficiency and utility in agricultural applications.

In this invention, a novel method was developed to enable soil bacteria to produce powerful chlorinated auxins such as 5-CI-IAA. A heterologous halogenase, such as L-tryptophan 5-halogenase, can be introduced into soil bacteria, including but not limited to Pseudomonas. The resulting strains can acquire the ability to produce chlorinated IAAs, which can be used in agriculture to stimulate crop growth and enhance food production. Therefore, this invention represents a novel and sustainable agricultural biotechnology that can increase food production without the need of chemical fertilizers, herbicides and pesticides that are harmful to the environment and human health.

### SUMMARY

A novel method was established to engineer soil bacteria for the ability to produce chlorinated auxins, which have strong plant growth-promoting ability and are not naturally produced by soil bacteria. Different L-tryptophan halogenases can be selected and introduced to soil bacteria for the production of specific chlorinated auxins. This method can be applied to a variety of soil microbes fit for different crops and environmental conditions, yielding a wide arsenal of chlorinated auxins-producing soil microbes for various crops and regions.

### BRIEF DESCRIPTION OF THE DRAWINGS

FIG. 1 illustrates the cloning and expression of PyrH in Escherichia coli BL21(DE3).

FIG. 2 shows the functional verification of PyrH in Escherichia coli BL21(DE3).

FIG. 3 shows the cloning of the pyrH gene into a Pseudomonas expression vector and analysis of the expression of PyrH in Pseudomonas putida KT2440.

FIG. 4 shows the HPLC analysis of 5-halogenation of L-tryptophan by Pseudomonas putida KT2440/pMiSI-pyrH.

FIG. 5 illustrates the conversion of IAA to 5-CI-IAA by Pseudomonas putida KT2440/pMiSI-pyrH.

FIG. 6 demonstrates the structural identification of the halogenated product of IAA by Pseudomonas putida KT2440/pMiSI-pyrH through NMR analysis.

FIG. 7 illustrates the HPLC analysis of direct production of 5-CI-IAA by Pseudomonas chlororaphis O6/pMiSI-pyrH.

### DETAILED DESCRIPTION

The present disclosure covers methods for constructing engineered strains of soil bacteria for the capability to produce chlorinated auxins. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, genes, structures, strains, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

In one embodiment, the present disclosure provides methods for engineering soil bacteria to produce chlorinated auxins such as 5-CI-IAA. By way of example, the present disclosure provides for introducing a microbial L-tryptophan 5-halogenase (PyrH) from Streptomyces rugosporus into two Pseudomonas strains to produce 5-CI-IAA. The methods described herein generally provide for a method to enable soil bacteria to produce chlorinated auxins that can better promote crop growth. Preferably, this method allows soil bacteria to produce chlorinated IAAs by incorporating a foreign halogenase, which will make soil bacteria more beneficial to crops and thus a useful agricultural biotechnology to enhance crop production. The details for the procedure are provided.

The present disclosure also provides for the methods to express useful enzymes in soil bacteria.

The genes encoding a L-tryptophan halogenase such as PyrH can be directly amplified from the genome or cDNA of a related microbial strain or chemically synthesized. The genes may be modified for improved activity or expression.

Expression of a L-tryptophan halogenase can be achieved in a variety of hosts such as Escherichia coli, Pseudomonas putida or other microbial strains. Any suitable bacterial strain, vector or culture condition may be used for the expression of L-tryptophan halogenases and production of chlorinated auxins. By way of example, suitable bacterial strains include Escherichia coli and two Pseudomonas strains. Alternatively, any species or strain of Pseudomonas may be used. Broadly, a suitable microbial strain is any soil bacterium strain capable of expressing a L-tryptophan halogenase. In some embodiments, chlorinated auxins may be generated by a microbial strains harboring a vector or vectors that encode for a L-tryptophan halogenase. The vector or vectors may be plasmids.

Chlorinated auxins may be produced by soil bacteria harboring a L-tryptophan halogenase from exogenously supplied substrates such as L-tryptophan and IAA. Chlorinated auxins may also be generated by soil bacteria harboring a L-tryptophan halogenase directly without supply of any said substrates.

The engineered microbial strains that harbor a L-tryptophan halogenase are grown in an appropriate medium. If there is an inducible promoter in the vector, a specific inducer will be added into the culture to induce protein expression. If a constitutive promoter is used, no inducer is needed. To produce a chlorinated auxin, such as 5-CI-IAA, substrates such as IAA will be added to the culture if the engineered strains don't naturally produce the substrate. No substrates are needed if the original soil bacteria have the ability to produce IAA.

The following examples are illustrative only and are not intended to limit the disclosure in any way. One skilled in the art would recognize various known methods and conditions for cloning or synthesizing a halogenase gene, expressing a halogenase in a soil bacterium, and analyzing the production of chlorinated IAAs. Each of these various embodiments are within the scope of the invention.

### EXAMPLES

The following materials and methods may be used in carrying out the various embodiments of the invention.

### Example 1. Bacterial Strains, Vectors, and Culture Conditions

Streptomyces rugosporus NRRL 21084 was obtained from USDA Agricultural Research Service Culture Collection. It was grown at 30° C. in YEME medium for the preparation of genomic DNA. Escherichia coli XL1-Blue and BL21(DE3) was purchased from Agilent. Pseudomonas putida KT2440 (ATCC 47054) was obtained from the American Type Culture Collection and Pseudomonas chlororaphis O6 was acquired from Dr. David Britt at Utah State University. Both Pseudomonas strains were routinely grown at 30° C. in LB medium.

Escherichia coli XL1-Blue was used for DNA cloning and amplification. Escherichia coli BL21(DE3) and pET28a (Novagen) were used for protein expression in Escherichia coli. Escherichia coli cells were grown in Luria-Bertani (LB) medium. pMiSI was used as the expression vector in Pseudomonas. When necessary, kanamycin was added into the culture medium at 50 μg/mL.

### Example 2. DNA Manipulations

The genomic DNA of Streptomyces rugosporus was isolated using standard methods. Plasmids in Escherichia coli were extracted using a GeneJET™ Plasmid Miniprep Kit (Fermentas).

### Example 3. Cloning of a L-Tryptophan 5-Halogenase Gene into pET28a

The pyrH gene (GenBank accession number AFV71318) was amplified from the genome of Streptomyces rugosporus via PCR using a set of specific primers, including 5′-AATTCATATGATTCGCAGCGTTGTGATTGTTG-3′ and 5′-AATTAAGCTTTTATTGAATACTGGCCAGGTATTC-3′. The Phusion high-fidelity DNA polymerase from Thermo Fisher Scientific was used. The PCR program used for amplification of the pyrH gene consisted of an initial denaturation at 98° C. for 5 minutes, 20 cycles of touchdown program (98° C. for 30 seconds, annealing at 70° C. for 40 seconds, decreasing 0.5° C. per cycle, and extension at 72° C. for 90 seconds), 20 cycles of regular program (98° C. for 30 seconds, annealing at 60° C. for 40 seconds, and extension at 72° C. for 90 seconds), followed by a final extension at 72° C. for 10 minutes.

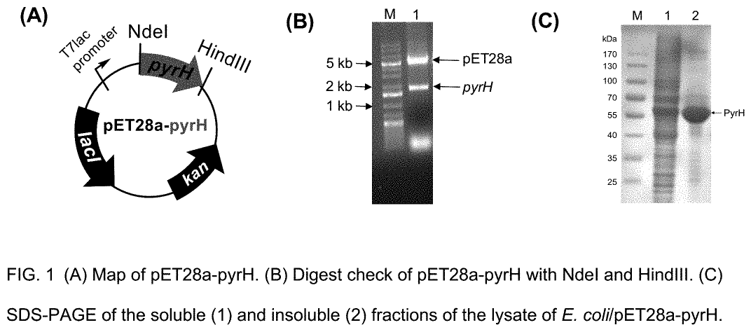

The PCR product was then digested with NdeI and HindIII at 37° C. for 2 hours, which was ligated into the pET28a expression vector between the NdeI and HindIII sites (FIG. 1A).

The ligation product was introduced into Escherichia coli XL1-Blue competent cells through chemical transformation. The transformants were grown on LB agar supplemented with 50 μg/mL kanamycin at 37° C. overnight.

Colonies were picked from the agar plate into 5 mL of LB broth with 50 μg/mL kanamycin at 37° C. and 250 rpm overnight. The plasmids were extracted and digested with NdeI and HindIII at 37° C. for 2 hours. The digestion of correct pET28a-pyrH is shown in FIG. 1B, which yielded the 1.6-kb insert and 5.3-kb vector.

### Example 4. Expression of PyrH in Escherichia coli BL21(DE3)

This new plasmid pET28a-pyrH was then introduced into Escherichia coli BL21(DE3) through chemical transformation. The transformants were grown on LB agar supplemented with 50 μg/mL kanamycin at 37° C. overnight.

A colony was picked from the agar plate into 5 mL of LB broth with 50 μg/mL kanamycin at 37° C. and 250 rpm. When the OD₆₀₀ value reached 0.5, isopropyl-β-D-1-thiogalactopyranoside (IPTG) was added into the culture at a final concentration of 200 μM. The culture was maintained at 28° C. and 250 rpm for 16 hours.

The cells were harvested by centrifugation at 4,000 rpm for 7 minutes. The cells were resuspended in 3 mL of lysis buffer (20 mM Tris-Cl, 500 mM NaCl, pH 7.9). After 10 minutes of ultrasonication (18 W, 30 s of interval), the resultant lysates were centrifuged at 20,000 rpm for 10 minutes. Insoluble proteins were dissolved in 8 M urea.

Both soluble and insoluble fractions were analyzed by 12% SDS-PAGE. As shown in FIG. 1C, PyrH (58.2 kDa) was seen in both soluble and insoluble fractions, confirming that this enzyme can be overexpressed in Escherichia coli through pET28a.

### Example 5. Functional Verification of PyrH in Escherichia coli BL21(DE3)

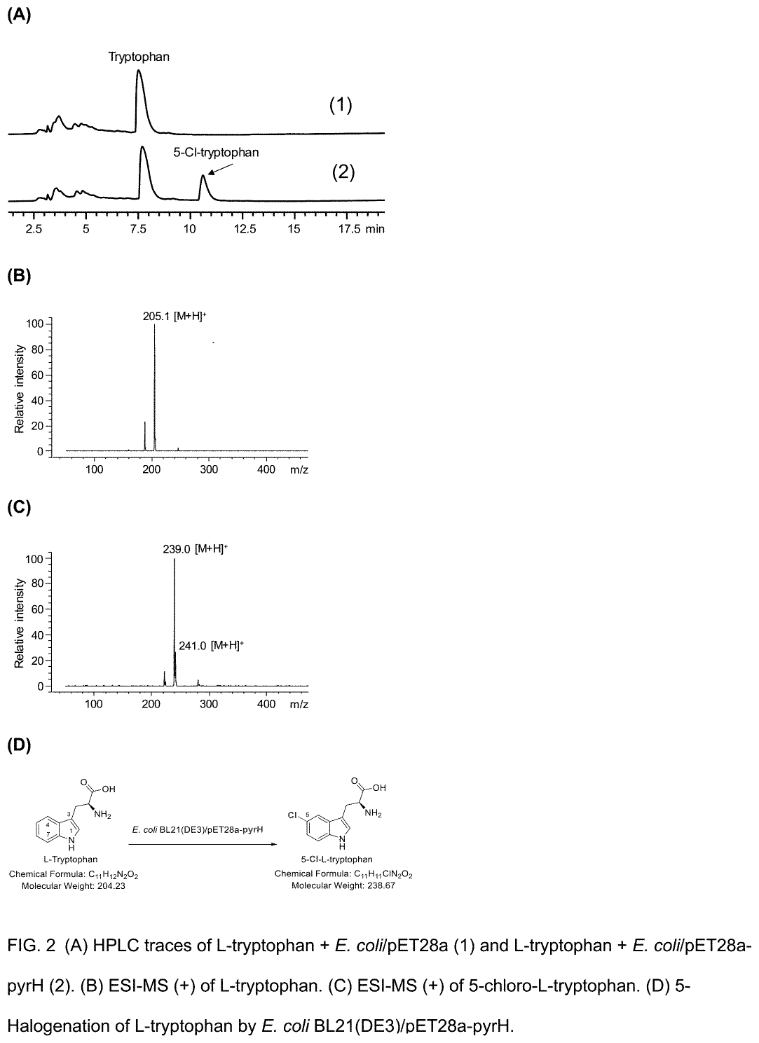

To test whether the expressed PyrH has the function of L-tryptophan 5-halogenase in Escherichia coli BL21(DE3), L-tryptophan was fed at 100 mg/L into the IPTG-induced broth of in Escherichia coli BL21(DE3)/pET28a-pyrH. After 1 day, 1 mL of the broth was taken from the culture and centrifuged at 15,000 rpm for 7 minutes. The supernatant was analyzed on an Agilent 6130 single quadrupole LC-MS equipped with an Agilent Eclipse XDB-C18 column (5 μm, 250 mm×4.6 mm). The sample was eluted with a gradient mobile phase of methanol-water (containing 0.1% formic acid) from 30% to 90% over 20 minutes at a flow rate of 1 mL/min. As shown in FIG. 2A, the substrate was converted to a less polar product. While L-tryptophan has a molecular weight of 204 (FIG. 2B), the new product has a molecular weight of 238, as revealed by the two [M+H]⁺ peaks at m/z 239.0 and 241.0 with a ratio of 3:1 (FIG. 2C) in the ESI-MS spectrum of showed, which is characteristic for monochlorinated compounds. Therefore, it can be confirmed that PyrH is functional in Escherichia coli as L-tryptophan 5-halogenase (FIG. 2D).

### Example 6. Cloning of a L-Tryptophan 5-Halogenase Gene into a Pseudomonas Expression Vector

Pseudomonas represents a most common family of soil bacteria. pMiSI is a Pseudomonas expression vector that has been used previously for expression of carotenoid biosynthetic genes. The pyrH gene was amplified from the genome of Streptomyces rugosporus via PCR using a set of specific primers, including 5′-AATTGTTTAAACATGATTCGCAGCGTTGTGATTGTTG-3′ and 5′-AATTGAAGCTTTTATTGAATACTGGCCAGGTATTC-3′. The Phusion high-fidelity DNA polymerase from Thermo Fisher Scientific was used. The PCR program used for amplification of the pyrH gene consisted of an initial denaturation at 98° C. for 5 minutes, 20 cycles of touchdown program (98° C. for 30 seconds, annealing at 70° C. for 40 seconds, decreasing 0.5° C. per cycle, and extension at 72° C. for 90 seconds), 20 cycles of regular program (98° C. for 30 seconds, annealing at 60° C. for 40 seconds, and extension at 72° C. for 90 seconds), followed by a final extension at 72° C. for 10 minutes.

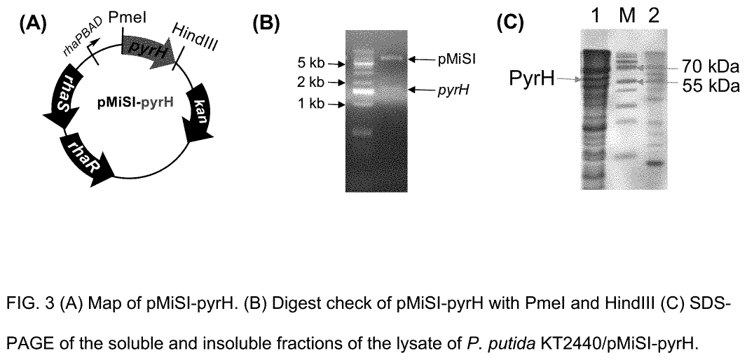

The PCR product was then digested with PmeI and HindIII at 37° C. for 2 hours, which was subsequently ligated into the pMiSI expression vector between the PmeI and HindIII sites (FIG. 3A).

The ligation product was introduced into Escherichia coli XL1-Blue competent cells through chemical transformation. The transformants were grown on LB agar supplemented with 50 μg/mL kanamycin at 37° C. overnight.

Colonies were picked from the agar plate into 5 mL of LB broth with 50 μg/mL kanamycin at 37° C. and 250 rpm overnight. The plasmids were extracted and digested with PmeI and HindIII at 37° C. for 2 hours. The digestion of correct pMiSI-pyrH is shown in FIG. 3B, which gave rise to the corresponding insert (1.6 kb) and vector (6.4 kb) bands.

### Example 7. Expression of PyrH in Pseudomonas putida KT2440

To test whether PyrH can be expressed in Pseudomonas, pMiSI-pyrH was introduced into the model soil bacterium Pseudomonas putida KT2440 through electroporation. The transformants were grown on LB agar with 50 μg/mL kanamycin at 30° C. overnight. L-Rhamnose was added at a concentration of 0.2% (w/v) to induce protein expression. After 36 hours, the cells were harvested by centrifugation at 4,000 rpm for 10 minutes. The cells were lysed as described in Example 4 and protein expression was analyzed by 12% SDS-PAGE. As shown in FIG. 3C, PyrH was successfully expressed in Pseudomonas putida KT2440.

### Example 8. Conversion of L-Tryptophan to 5-CI-Tryptophan by Pseudomonas putida KT2440/pMiSI-pyrH

Although SDS-PAGE analysis indicated that PyrH was expressed in Pseudomonas putida KT2440, it is unclear whether the L-tryptophan 5-halogenase is functional in this soil bacterium. To test this, L-tryptophan was added into the fermentation broth of P. putida KT2440/pMiSI-pyrH at 100 mg/L.

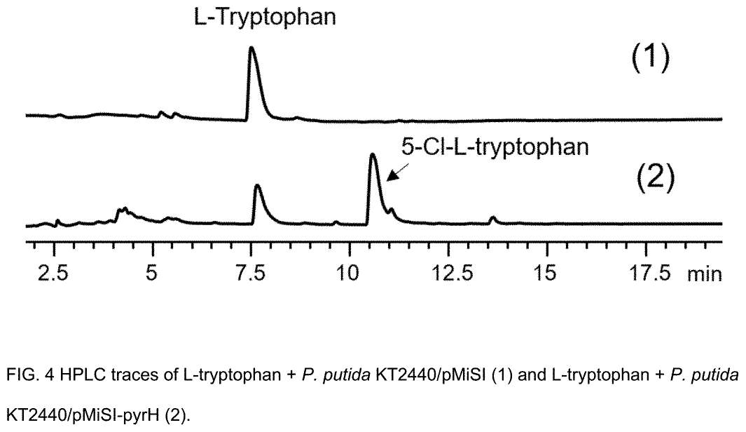

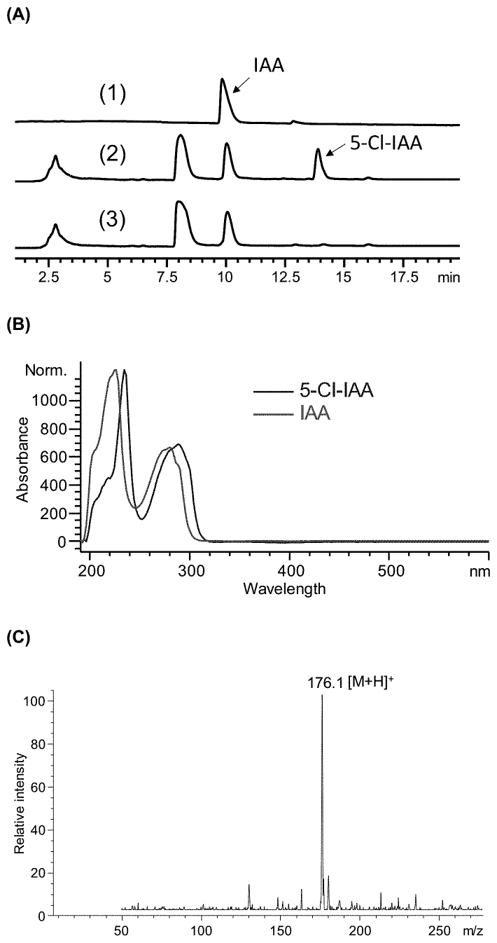

After 36 hours, 1 mL of the broth was taken from the culture and centrifuged at 15,000 rpm for 7 minutes. The supernatant was analyzed on an Agilent 1200 HPLC equipped with an Agilent Eclipse XDB-018 column (5 μm, 250 mm×4.6 mm). The sample was eluted with a gradient mobile phase of methanol-water (containing 0.1% formic acid) from 30% to 90% over 20 minutes at a flow rate of 1 mL/min. The broth was analyzed by HPLC. As shown in FIG. 4, L-tryptophan was successfully converted to 5-CI-tryptophan by Pseudomonas putida KT2440/pMiSI-pyrH.

### Example 9. Production of 5-CI-IAA from Exogenous IAA by Pseudomonas putida KT2440/pMiSI-pyrH

IAA is a most common and widely used plant auxin. It is known that chlorinated derivatives of IAA are much effective than IAA in promoting plant growth. Because IAA shares the same indole ring as L-tryptophan, it is of interest to test whether P. putida KT2440/pMiSI-pyrH has relaxed substrate specificity and can halogenate IAA to yield a more powerful auxin. To this end, I supplied IAA into the rhamnose-induced broth of this engineered strain.

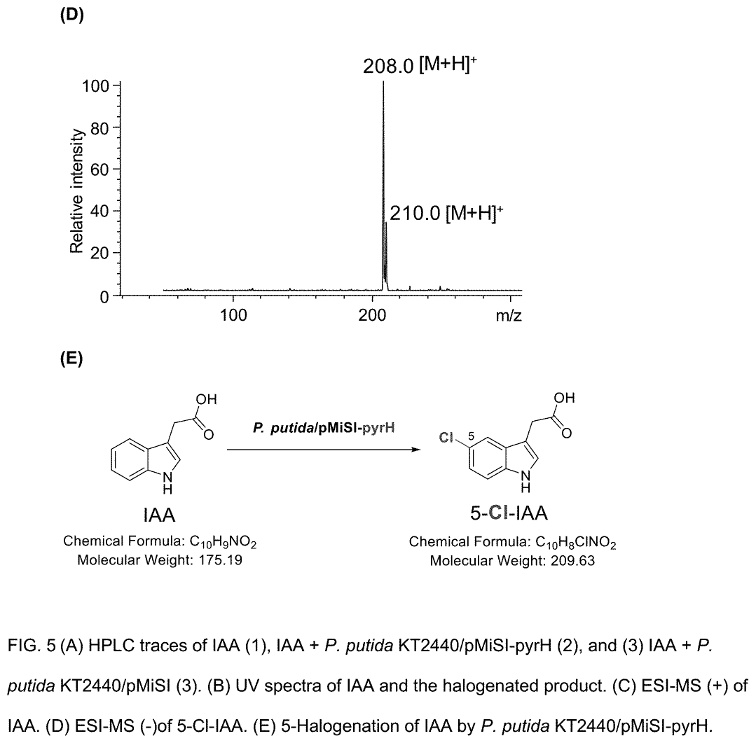

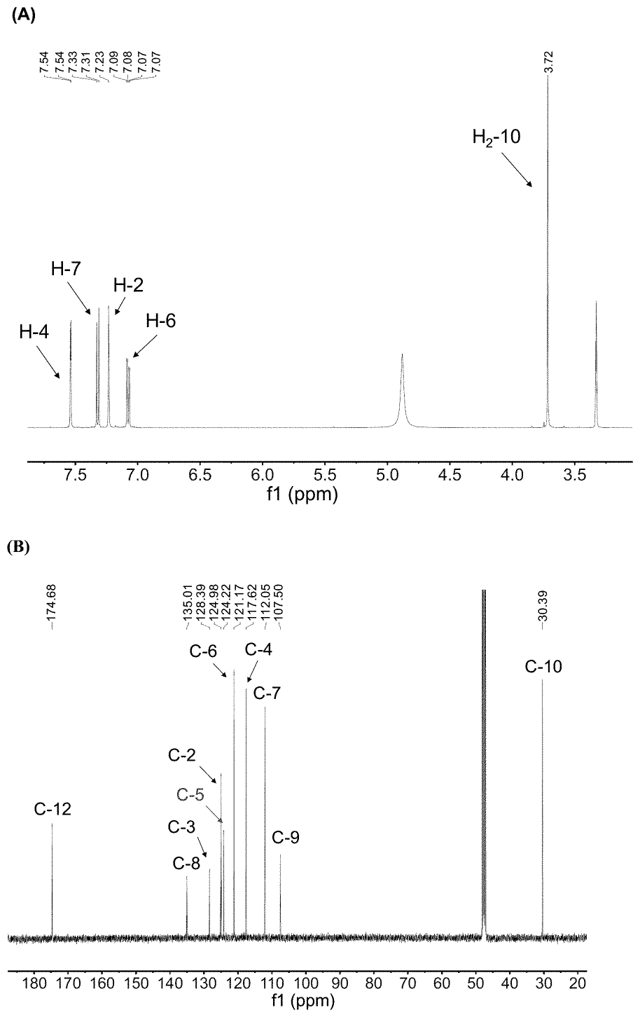

As shown in FIG. 5A, P. putida KT2440/pMiSI-pyrH converted IAA into a less polar product with a retention time of 13.8 minutes. By contrast, no product was observed for Pseudomonas putida KT2440 harboring the empty pMiSI vector. A comparison of the UV spectra of IAA and the product (FIG. 5B) showed a bathochromic shift of the maximum UV absorptions, suggesting that IAA was chlorinated. Further ESI-MS analysis (FIGS. 5C and 5D) indicated that the product has a molecular weight of 209, which is 34 mass units larger than IAA. The two [M−H]⁻ peaks at m/z 208.0 and 210.0 (3:1) further indicated that this product is a monochlorinated derivative of IAA (FIG. 5E).

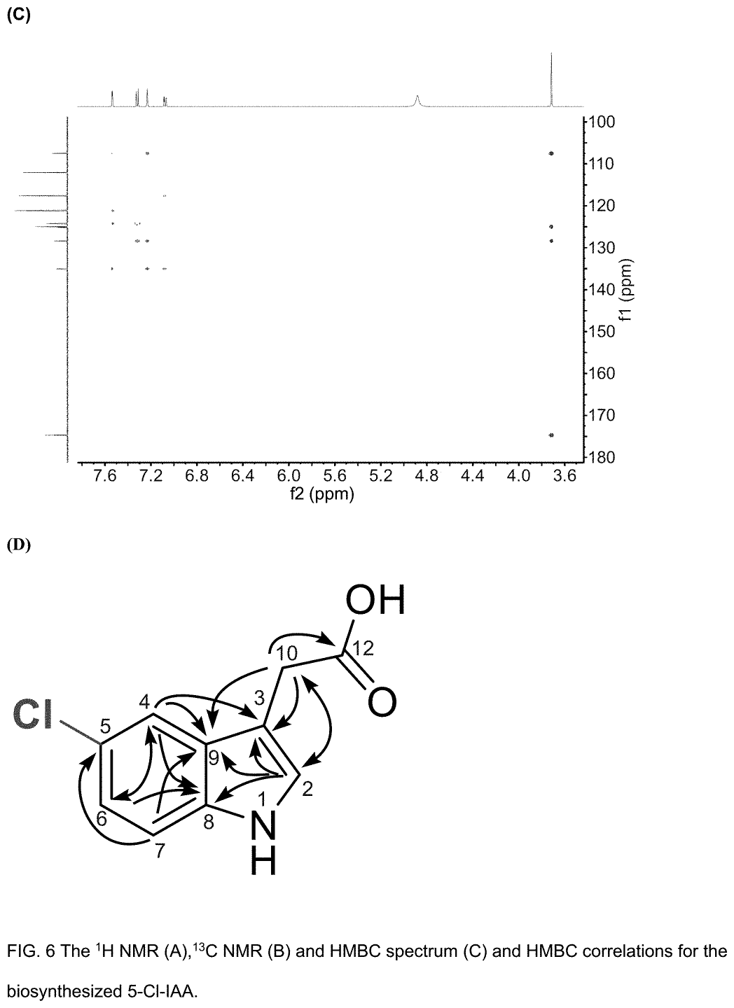

To confirm that the halogenation really occurred at C-5 of IAA, the product was purified from the broth and collected its 1D and 2D NMR spectra. The ¹H, ¹³C and HMBC NMR spectra are shown in FIGS. 6A-6C, respectively. The summarized HMBC correlations are given in FIG. 6D, which confirmed that the product is indeed 5-CI-IAA. All the proton and carbon signals were assigned and are marked in FIGS. 6A and 6B.

### Example 10. Engineering of the Crop-Benefiting Soil Bacterium Pseudomonas chlororaphis O6 for Direct Production of 5-CI-IAA

Pseudomonas chlororaphis O6 is a crop-benefiting soil bacterium that was known to colonize the roots of crops such as wheat. It was found to produce IAA. To enable soil bacteria to directly produce 5-CI-IAA without exogenous supply of IAA, the pMiSI-pyrH plasmid was introduced into Pseudomonas chlororaphis O6 by electroporation. The correct transformant of Pseudomonas chlororaphis O6/pMiSI-pyrH was selected on LB agar with 50 μg/mL kanamycin.

Pseudomonas chlororaphis O6/pMiSI-pyrH was grown in 50 mL of LB broth with 50 μg/mL kanamycin at 30° C. and 250 rpm. L-Rhamnose was added at a final concentration of 0.2% (w/v) to induce protein expression. Wild type Pseudomonas chlororaphis O6 was grown in LB broth as the control. After 36 hours, 1 mL of broth was taken from these cultures and centrifuged at 15,000 rpm for 7 minutes. The supernatants were analyzed on an Agilent 1200 HPLC equipped with an Agilent Eclipse XDB-C18 column (5 μm, 250 mm×4.6 mm). The sample was eluted with a gradient mobile phase of methanol-water containing 0.1% formic acid from 30% to 90% over 20 minutes at a flow rate of 1 mL/min. the broth was analyzed by HPLC.

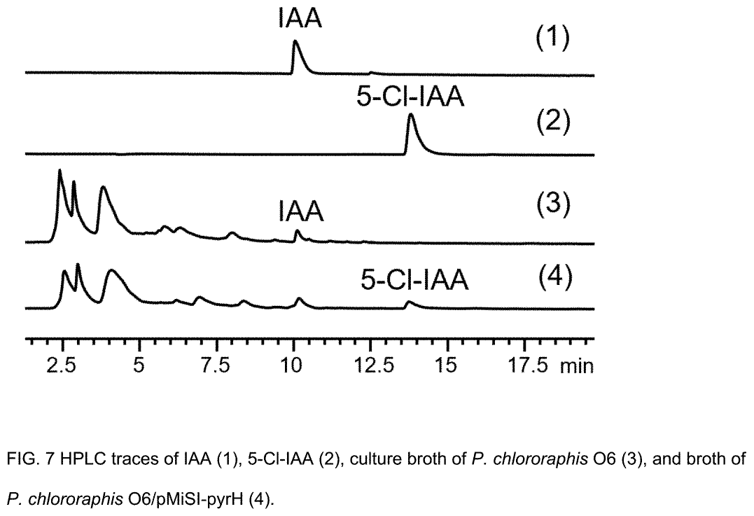

As shown in FIG. 7, compared to the standards of IAA (trace 1) and 5-CI-IAA (trace 2), the fermentation broth of Pseudomonas.chlororaphis O6 produced IAA as a metabolite (trace 3), and Pseudomonas chlororaphis O6/pMiSI-pyrH produced 5-CI-IAA (trace 4). Therefore, the engineered Pseudomonas chlororaphis O6 strain can directly produce 5-CI-IAA without adding IAA as the substrate.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

### NON-PATENT CITATIONS

Simon S, Kubeš M, Baster P, Robert S, Dobrev P I, Friml J, Petrášek J, Zažímalová E. Defining the selectivity of processes along the auxin response chain: a study using auxin analogues. New Phytologist 2013, 200:1034-1048.
Wright M, Adams J, Yang K, McManus P, Jacobson A, Gade A, McLean J, Britt D, Anderson A. A root-colonizing Pseudomonad lessens stress responses in wheat imposed by CuO nanoparticles. PLoS One 2016, 11:e0164635.
Reinecke D M. 4-Chloroindole-3-acetic acid and plant growth. Plant Growth Regulation 1999, 27:3-13.
Leyser O. The power of auxin in plants. Plant Physiology 2010, 154:501-505.
Enders T A, Strader L C. Auxin activity: Past, present, and future. American Journal of Botany 2015, 102:180-196
Wang Y, Zhang T, Wang R, Zhao Y. Recent advances in auxin research in rice and their implications for crop improvement. Journal of Experimental Botany 2018, 69:255-263.
Zhu X, De Laurentis W, Leang K, Herrmann J, Ihlefeld K, van Pée K H, Naismith J H. Structural insights into regioselectivity in the enzymatic chlorination of tryptophan. Journal of Molecular Biology 2009, 391:74-85.
Teale W D, Paponov I A, Palme K. Auxin in action: signalling, transport and the control of plant growth and development. Nature Reviews Molecular Cell Biology 2006, 7:847-859.
Böttger M, Engvild K C, Soll H. Growth of Avena coleoptiles and pH drop of protoplast suspensions induced by chlorinated indoleacetic acids. Planta 1978, 140:89-92.
Zhao Y. Auxin biosynthesis and its role in plant development. Annual Review of Plant Biology 2010, 61:49-64.
Chandra S, Askari K, Kumari M. Optimization of indole acetic acid production by isolated bacteria from Stevia rebaudiana rhizosphere and its effects on plant growth. Journal of Genetic Engineering and Biotechnology 2018, 16:581-586.
Martins dos Santos V A P, Timmis K N, Tümmler B, Weinel C. (2004) Genomic features of Pseudomonas putida strain KT2440. In: Ramos J L. (eds) Pseudomonas. Springer, Boston, Mass.
Mi J, Becher D, Lubuta P, Dany S, Tusch K, Schewe H, Buchhaupt M, Schrader J. De novo production of the monoterpenoid geranic acid by metabolically engineered Pseudomonas putida. Microbial Cell Factories 2014, 13:170.
Shah S H. Comparative effects of 4-CI-IAA and kinetin on photosynthesis, nitrogen metabolism and yield of black cumin (Nigella sativa L.). Acta Botanica Croatica 2011, 70:91-97.
Katznelson H, Sirois J C. Auxin production by species of Arthrobacter. Nature 1961, 191:1323-1324.
Lam H K, Ross J J, McAdam E L, McAdam S A M. The single evolutionary origin of chlorinated auxin provides a phylogenetically informative trait in the Fabaceae. Plant Signaling & Behavior 2016, 11:e1197467.

What is claimed is:

1. A method of constructing soil bacterial strains capable of producing chlorinated auxins. The said method comprises:
(a) obtaining a tryptophan halogenase gene through gene synthesis, PCR amplification, or digest of genomic DNA;
(b) introducing a tryptophan halogenase into soil bacteria;
(c) testing the ability of engineered strains to produce chlorinated derivatives of indole-3-acetic acid (IAA) in the presence or absence of L-tryptophan or IAA.

2. The method of claim 1 further comprises the use of a natural, recombinant, or synthesized tryptophan halogenase such as PyrH in soil bacteria. The said PyrH has an amino acid sequence shown in SEQ ID NO. 1.

3. The method of claim 1 further comprising an expression system of halogenases with inducible or constitutive promoter in soil bacteria. The expression can be achieved through an expression plasmid or insertion of the target halogenase gene into the genome of soil bacteria.
